(19) United States
(12) Patent Application Publication
(10) Pub. No.: US 2022/0049298 A1
(43) Pub. Date: Feb. 17, 2022

(54) TAGGED-BASE DNA SEQUENCING READOUT ON WAVEGUIDE SURFACES

(71) Applicant: SiPhox, Inc., Burlington, MA (US)

(72) Inventors: Michael Dubrovsky, Somerville, MA (US); Diedrik Rene Vermeulen, Somerville, MA (US)

(73) Assignee: SiPhox, Inc., Burlington, MA (US)

(21) Appl. No.: 17/403,480

(22) Filed: Aug. 16, 2021

Related U.S. Application Data
(60) Provisional application No. 63/065,611, filed on Aug. 14, 2020.

(51) Int. Cl. C12Q 1/6869; G01N 21/77

(52) U.S. Cl. CPC C12Q 1/6869; G01N 2021/7786; G01N 2021/7783; G01N 21/77

(57) ABSTRACT

A method of tagged-base DNA sequencing readout on waveguide surfaces includes immobilizing, a surface of a waveguide, a nucleotide fragment, exposing the nucleotide fragment to a first plurality of capped nucleotides, wherein the first plurality of capped nucleotides include a first plurality of nucleotide types, each distinct nucleotide type has a distinct capping agent, and each distinct capping agent has a distinct optical signature, severing base pair connections between the at least a nucleotide fragment and the first plurality of capped nucleotides, wherein the nucleotide fragment remains attached and a first single nucleotide, of the first plurality of capped nucleotides, remains immobilized on a nucleotide binding locus adjacent to the first nucleotide sequence, and detecting a first distinct optical signature of a first distinct capping agent of the first single nucleotide using the waveguide.

### CROSS-REFERENCE TO RELATED APPLICATIONS

This application claims the benefit of priority of U.S. Provisional Patent Application Ser. No. 63/065,611, filed on Aug. 14, 2020, and titled “Tagged-Base DNA Sequencing Readout on Waveguide Surfaces,” which is incorporated by reference herein in its entirety.

### FIELD OF THE INVENTION

The present invention generally relates to the field of on-chip chemical reactions. In particular, the present invention is directed to tagged-base DNA sequencing readout on waveguide surfaces.

### BACKGROUND

Many nucleotide sequencing methods use fluorescently labeled nucleotides to track the copying of nucleotide sequences in order to extract the original sequence. However, these methods currently require expensive optics.

### SUMMARY OF THE DISCLOSURE

In an aspect, a method of tagged-base DNA sequencing readout on waveguide surfaces includes immobilizing, a surface of a waveguide, a nucleotide fragment, exposing the nucleotide fragment to a first plurality of capped nucleotides, wherein the first plurality of capped nucleotides include a first plurality of nucleotide types, each distinct nucleotide type has a distinct capping agent, and each distinct capping agent has a distinct optical signature, severing base pair connections between the at least a nucleotide fragment and the first plurality of capped nucleotides, wherein the nucleotide fragment remains attached and a first single nucleotide, of the first plurality of capped nucleotides, remains immobilized on a nucleotide binding locus adjacent to the first nucleotide sequence, and detecting a first distinct optical signature of a first distinct capping agent of the first single nucleotide using the waveguide.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a schematic diagram of an exemplary embodiment of a system for tagged-base DNA sequencing;

FIG. 2 is a schematic diagram of an exemplary embodiment of a system for tagged-base DNA sequencing;

FIG. 3 is a flow diagram illustrating an exemplary method of tagged-base DNA sequencing;

FIG. 4 is a schematic diagram of an exemplary embodiment of detection optics for tagged-base DNA sequencing;

FIG. 5 is a schematic diagram of an exemplary embodiment of detection optics for tagged-base DNA sequencing;

FIG. 6 is a schematic diagram of an exemplary embodiment of detection optics for tagged-base DNA sequencing;

FIG. 7A-C are schematic diagrams of an exemplary embodiment of detection optics for tagged-base DNA sequencing; and

FIG. 8 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

Generally, the present disclosure discusses methods of labelling nucleotides for detection on the surface of a waveguide as well as methods for detecting nucleotides using waveguides. Deoxyribonucleic acid (DNA) and/or ribonucleic acid (RNA) sequencing can be accomplished by monitoring growth of complimentary DNA and/or RNA strands that are immobilized on one or more surfaces. The present disclosure describes a novel method of monitoring an optical readout associated with growth of nucleotide sequence using a waveguide, which also acts as the solid support for the DNA or RNA. Additionally, methods to tag nucleotides with optically active agents (absorbers, fluorophores, etc.) are described that may permit monitoring of nucleotide growth via optical readout. For example, each type of nucleotide may have a different tag with a different absorbance spectrum to allow for the monitoring of the growth of the chain.

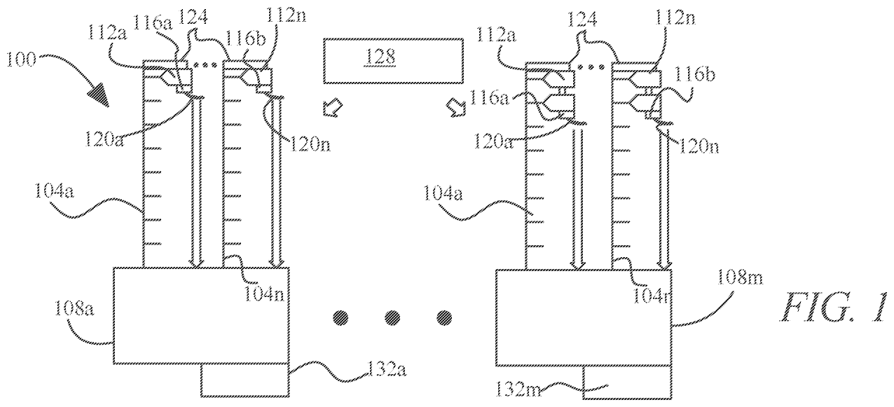

Referring now to FIG. 1, an exemplary embodiment of a system 100 for tagged-base DNA sequencing readout on waveguide surfaces is illustrated. In a nonlimiting example, single nucleotide fragments 104a-n such as fragments of DNA and/or RNA may be attached to a surface, which may be a surface of one or more waveguides 108a-m; fragments may be obtained, without limitation, as described below. Such fragments may be locally amplified as described in further detail below, for instance and without limitation via bridge amplification polymerase chain reaction (PCR), while surface bound into clusters of identical sequences. In some cases, for instance as described below sequences may be copied using polymerase and terminating nucleotides having capping agents a-n may be appended. Terminating nucleotides prevent further addition of nucleotides until capping agents a-n are removed from the nucleotide in a deprotecting or decapping step; this may have the effect, in a non-liming example, of limiting buildup of one nucleotide per cycle on a replicated sequence. Each capping agent a-n may have a signature element 120a-n, as described in further detail below, which may evince a nucleotide type-specific optical signature as described in further detail below; for instance and without limitation, each capping agent a-n may contain a different fluorophore. Thus, each cluster of identical sequences, after any given process step as described in this disclosure, may have a distinct optical signature, such as a different fluorescence color, when excited corresponding to the appropriate fluorophore on the terminating nucleotide that was just added to the sequence. By measuring the fluorescence and/or other optical signature of each cluster at each step, a sequence of each cluster may be inferred. This may permit reconstruction of the entire original genome from the collected optical data, for instance and without limitation as described in further detail below.

Still referring to FIG. 1, in embodiments, and as described in further detail below, a chain or sequence of nucleotides to be appended with capping agents a-n and/or signature elements 120a-n as described above may be initiated at a binding locus 124. A “binding locus,” as used in this disclosure, is an element to which a first nucleotide may be attached using, as a non-limiting example, an enzyme such as DNA or RNA polymerase. A binding locus 124 may include, without limitation, a primer attached to a nucleotide sequence that is part of a nucleotide fragment 104a-n; such nucleotide sequence may be appended to a nucleotide fragment 104a-n, for instance and without limitation during a bridge polymerase chain reaction (PCR) process or the like. A primer may include, without limitation, a synthetic DNA and/or RNA oligonucleotide, which may include for instance approximately 15-30 bases. A primer may be designed to bind, via sequence complementarity, to sequences at one extreme or another of a sequence of interest such as a fragment to be sequenced. As such, primers' binding sites may be unique to the vicinity of the target with minimal homology to other sequences of fragment. Uniqueness may, without limitation, be determined statistically by use of a primer having a predefined sequence and sufficient length to make similar and/or complementary sequences improbable. Alternatively or additionally, a first primer candidate may be capped with a signature element 120a-n, exposed to nucleotide fragment 104a-n, and subsequently washed, where nucleotide fragment 104a-n includes an element that is complementary to first primer candidate, an optical signature thereof may be detected indicating at least one such binding location. In some embodiments, upon such detection an alternative or second primer candidate may be selected, this process may be repeated until a suitable primer candidate is discovered, after which a complementary sequence may be appended to nucleotide fragment 104a-n, and clones thereof, during bridge PCR processes.

With continued reference to FIG. 1, capped nucleotides a-n may be provided in the form of nucleotide triphosphates. For instance, capped nucleotides a-n may be provided as: deoxyribose nucleotide triphosphates (dNTPs) and/or ribose nucleotide triphosphates (rNTPs) attached to capping agents a-n. As a non-limiting example, dNTPs may be provided for four basic nucleotides, in the form of deoxyadenosine triphosphate (dATP), deoxycytidine triphosphate (dCTP), deoxyguanosine triphosphate (dGTP), and deoxythymidine triphosphate (dTTP), corresponding respectively to the nucleotides adenine, cytosine, guanine, and thymine. These four nucleotides may be provided in approximately equimolar amounts. NTPs corresponding to all four RNA nucleotides, adenine, cytosine, guanine, and uracil, which may be provided in approximately equimolar amounts. Alternatively or additionally, a capped nucleotide may include a nucleobase paired to a peptide which may be appended to a peptide backbone using a polymerase or other enzyme.

Still referring to FIG. 1, capping agents a-n may act to prevent formation of covalent bonds between capped nucleotides a-n; as a result, each addition step may permit only addition of a single capped nucleotide to a binding locus 124 and/or to a nucleotide added in a previous step. Moreover, the single capped nucleotide to be added may be a nucleotide that has formed a base pair with a nucleotide adjacent in nucleotide fragment 104a-n to a base pair including a previously added nucleotide and/or a binding location; this may ensure that each added capped nucleotide complements a proximate nucleotide in an order of traversal of nucleotide fragment 104a-n, ensuring an accurate sequencing analysis thereof.

With continued reference to FIG. 1, a capped nucleotide may be added to a binding locus 124 and/or previously appended nucleotide using a catalyst, which may include an enzyme. Enzymes used to form covalent bonds between nucleotides may include, without limitation, DNA polymerase such as without limitation Taq polymerase. Polymerase may be included in solution with capped nucleotides a-n and/or one or more buffering and/or activating agents. Activating agents may include, without limitation, magnesium ion (Mg²⁺), which may function as a cofactor for activity of DNA polymerases by enabling incorporation of dNTPs during polymerization. Buffering agents may create a buffer that provides a suitable chemical environment for activity of DNA polymerase. Buffer pH may be in any suitable range, including between 8.0 and 9.5; buffering solution may be stabilized by Tris-HCl. Buffering agents may include, without limitation, potassium ion (K⁺) from KCl, ammonium sulfate (NH₄)₂SO₄, or the like.

Still referring to FIG. 1, one or more capped nucleotides may be added in a time-multiplexed manner; for instance, a solution containing only a first capped nucleotide type, such as only thymine and/or a thymine-containing compound such as dTTP may be added to nucleotide fragment, polymerized, have base pairs severed, and then be washed away, after which a solution containing only a second capped nucleotide type such as guanine and/or a guanine-containing compound such as dGTP may be added to nucleotide fragment, polymerized, have base pairs severed, and then be washed away and so forth. After each addition, a detection process may be performed as described below to determine if a capped nucleotide was added In this case, each type of capped nucleotide may have a different optical signature; alternatively or additionally, all capped nucleotides may have the same optical signature and/or optical signatures may not distinguish between types, which may be distinguished instead by a detection step after each addition and washing step.

Further referring to FIG. 1, a capping agent may include a modified polymerase which is affixed to a nucleotide and/or nucleotide containing compound such as a dNTP and/or NTP, and/or which affixes itself thereto to form a capped nucleotide; such modified polymerase may be able only to connect a single nucleotide to a chain such as a sequence complementary to nucleotide fragment. Thus, a capped nucleotide complementary to a subsequent nucleotide of nucleotide fragment to be detected may be added using its modified polymerase as soon as it forms a base pair adjacent to binding locus and/or a chain bound thereto. A modified polymerase may be tagged with any signature element described above; alternatively or additionally, modified polymerase may be a signature element itself. For instance, waveguide may 108a-m may be used to detect polymerase using a refractive index change caused by the relatively large volume of the polymerase molecule. As a non-limiting example, after bridge amplification on top of microring resonators as described below, such a polymerase may be detected binding to a growing dna copy using resonance of the microring resonator. In an embodiment, decapping may removes a modified polymerase; for instance, if the enzyme is linked to a particular base, when the enzyme adds that base to the growing copy, it may become stuck there until it is cleaved off.

Still referring to FIG. 1, waveguide 108a-m may also act as a surface support for immobilization of fragments and/or sequence buildup; this may permit collection of optical signatures via the waveguide 108a-m and substrate, rather than collection from out-of-plane. In an embodiment, and as shown in FIG. 1, an out-of-plane light source may be used. Out-of-plane light source may include any suitable light source as described in further detail below. Light may be received at one or more detectors 132a-m, and analyzed by circuits connected thereto. Waveguide, waveguide surfaces, detection methods used therewith, and/or methods for affixing and/or immobilizing materials thereon may be implemented without limitation as disclosed in U.S. Nonprovisional patent application Ser. No. 17/337,931, filed on Jun. 3, 2021 and entitled “METHODS AND SYSTEMS FOR MONOMER CHAIN FORMATION,” the entirety of which is incorporated herein by reference.

Further referring to FIG. 1, system 100 may include a light source configured to emit a light, which may be emitted into and/or at waveguide 108a-m. As used in this disclosure, a “light source” is any device configured to emit a light. A light source may include a coherent light source and/or an incoherent light source. Non-limiting exemplary light sources include lasers, light emitting diodes (LEDs), organic LEDs (OLEDS), light emitting capacitors, incandescent lamps, fluorescent lamps, and the like. Light sources may include lasers, such as diode and/or grating-based lasers, including without limitation tunable lasers. Light source may include broad-band light sources for detection of absorbent signature elements 120a-n as described in further detail below. Light source may be coupled into waveguide 108a-m and propagate within waveguide 108a-m, for example and without limitation using total internal reflection.

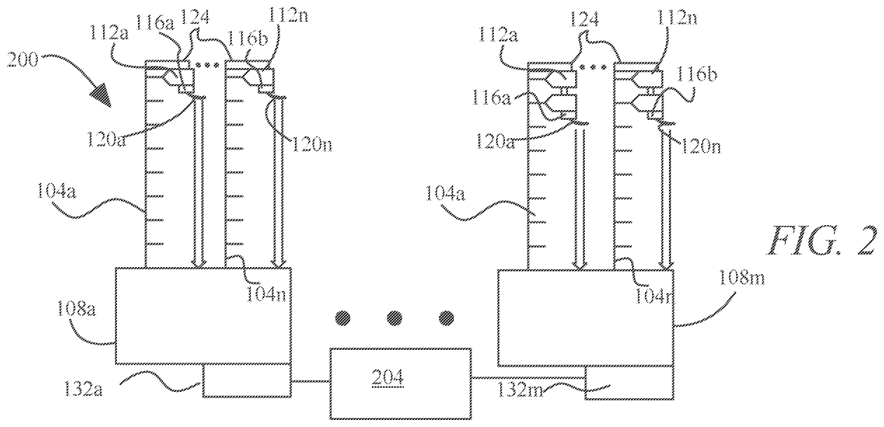

Alternatively or additionally, and referring to FIG. 2, light may be provided to capping agents a-n and/or signature elements 120a-n from and/or using waveguide 108a-m. Provision using waveguide 108a-m may be performed by optically connecting any light source as described herein to waveguide 108a-m. Light from optical source may be provided using any suitable light source as described in this disclosure.

Still referring to FIG. 2, signature element 120a-n may include any substance, compound, nanoparticle, or the like having a detectable effect on light received at one or more detectors 132a-m. For instance, and without limitation, signature element 120a-n may include one or more fluorescent elements such as fluorophores; each nucleotide type may be capped with a fluorescent element emitting a distinct wavelength, such that, for instance, a wavelength emitted by a capped adenine is measurably different from a wavelength emitted by a capped guanine, cytosine, thymine, and/or uracil, which may further be measurably different from each other.

With continued reference to FIG. 2, a similar optical readout scheme may be accomplished by a scheme a capping agent a-n may be tagged with an optical absorber. As broadband light, such as without limitation a swept laser source, passes through the waveguide 108a-m, broadband light may be absorbed by an optical absorber and the loss of signal, or attenuation, may be monitored by detection and/or analysis of a spectrum of light received at detectors 132a-m. Choosing different optical absorbers for each nucleotide may allow minimum overlap between each unique nucleotide and may result in a uniquely identifiable absorbance tag. In some examples, silicon nitride or other waveguide 108a-m materials with broad transmission windows may be used to make it easier to separate absorption and/or emission windows of signature elements 120a-n.

Still referring to FIG. 2, methods and systems described in this disclosure may be used to track any polymerization reaction, and may be combined with other methods for DNA or RNA sequencing that use tagged nucleotides immobilized on a surface. Waveguides 108a-m, sensing chips, and/or fibers may be made using silicon, silicon nitride, silicon dioxide and/or any other commonly used waveguide 108a-m materials.

Still referring to FIG. 2, in some embodiments, system 100 may additionally include a microfluidic channel (not shown). Microfluidic channel may be configured to permit a solution to flow through microfluidic channel and/or to store a solution. In some cases, solution may include any chemical agents described above, including capped nucleotides a-n, primers, polymerase, activating chemicals, buffers, or the like. As used in this disclosure, a “microfluidic channel” is a fluidic pathway having a characteristic width less than 10 mm. A microfluidic channel may be consistent with channels used for fluidic communication in microfluidic chips and/or microfluidic circuits. In some cases, a fluidic channel of any size may be configured to permit a solution to flow through microfluidic channel and/or to store a solution. In some cases, a sample fluid may be split up into separate chambers, each containing a different chemical agent for a different stage in processes described in this disclosure. Microfluidics may alternatively or additionally be used to wash away removed and/or unattached capped nucleotides a-n and/or capping agents a-n prior to a next step to ensure effective sensing, addition, and de-capping steps.

Continuing to refer to FIG. 2, system 100 may include a control circuit 204. Control circuit 204 may control one or more components and/or devices, such as valves, pipettes, microfluidic channels, valves, or the like, light sources such as without limitation lasers, LEDS, or the like, sensors, and/or any other components for accomplishing any step described in this disclosure and/or any related step that may occur to persons skilled in the art upon reviewing the entirety of this disclosure. Control circuit 204 may alternatively or additionally perform amplification and/or analysis of detected optical signatures.

In some cases, and with further reference to FIG. 2, a photodetector 132a-m may be communicative with a circuit, for example without limitation an analog circuit. Circuit may take as input a signal from photodetector 132a-m and process the signal. In some cases, one or more optical elements and/or optical systems may be used to couple light into and/or out from waveguide 108a-m. For example, coupling lenses having a numerical aperture selected based upon acceptable entrance angle and/or cross-sectional area of waveguide 108a-m may be used to couple substantially collimated light into waveguide 108a-m and/or substantially collimate light after exiting the waveguide 108a-m. Circuit may include analog and/or digital circuit elements. Exemplary non-limiting analog elements include operational amplifiers, comparators, amplification circuits, and the like. In some cases, circuit may include an analog circuit interfaced with a digital circuit, for example without limitation by way of an analog to digital (A/D) converter. Analog circuit may include one or more operational amplifiers, which may be used, without limitation, to amplify an electrical signal from a photodetector 132a-m. Alternatively or additionally, an analog circuit may be interfaced with a digital circuit by way of a resistive divider, such as without limitation a Wheatstone bridge. Alternatively or additionally, analog circuit may be interfaced with a digital circuit by way of at least a control terminal of transistors (or other digital elements), which are configured to trip (or otherwise digitally indicate) a certain voltage threshold configured to be indicative of a change in digital state. In some cases, circuit may include a digital circuit. Digital circuit could be any combinatorial or sequential circuit including logic gates, registers, and the like. Digital circuit may include a microprocessor, microcontroller, or the like. Digital circuit may include connection to a memory. In some embodiments, digital circuit may include or interface with at least a computing device. Computing device may include any computing device described in this disclosure. In some cases, system 100 may be configured with aid of a computing device to perform any methods, steps, and/or processes described in this disclosure automatically.

Still referring to FIG. 2, a computing device may include any computing device as described in this disclosure, including without limitation a microcontroller, microprocessor, digital signal processor (DSP) and/or system on a chip (SoC) as described in this disclosure. A computing device may include, be included in, and/or communicate with a mobile device such as a mobile telephone or smartphone. A computing device may include a single computing device operating independently, or may include two or more computing device operating in concert, in parallel, sequentially or the like; two or more computing devices may be included together in a single computing device or in two or more computing devices. A computing device may interface or communicate with one or more additional devices as described below in further detail via a network interface device. Network interface device may be utilized for connecting a computing device to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. A computing device may include but is not limited to, for example, a computing device or cluster of computing devices in a first location and a second computing device or cluster of computing devices in a second location. A computing device may include one or more computing devices dedicated to data storage, security, distribution of traffic for load balancing, and the like. A computing device may distribute one or more computing tasks as described below across a plurality of computing devices of computing device, which may operate in parallel, in series, redundantly, or in any other manner used for distribution of tasks or memory between computing devices.

With continued reference to FIG. 1, computing device and/or control circuit 204 may be designed and/or configured to perform any method, method step, or sequence of method steps in any embodiment described in this disclosure, in any order and with any degree of repetition. For instance, computing device and/or control circuit 204 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device and/or control circuit 204 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

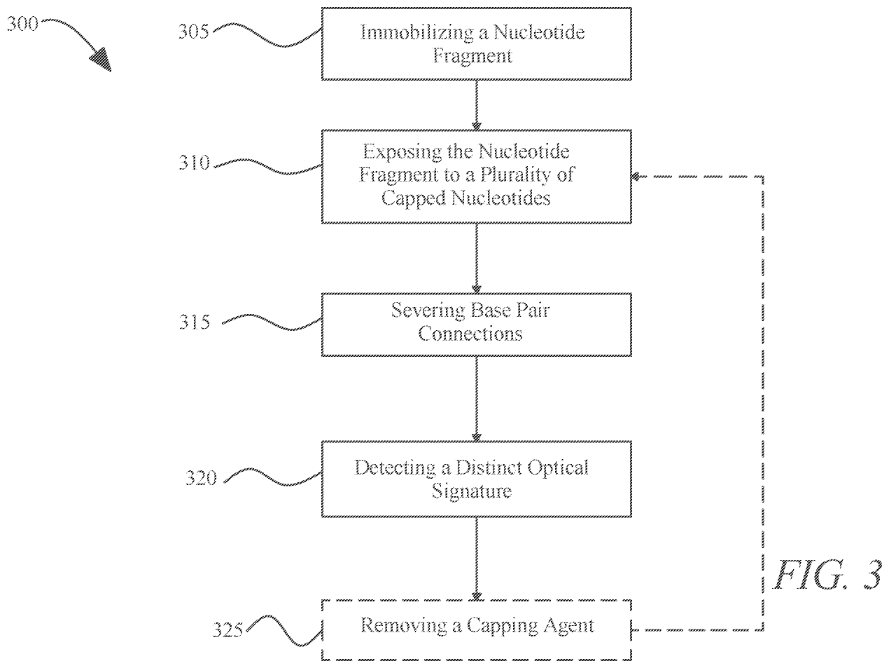

Referring now to FIG. 3, an exemplary embodiment of a method 300 of tagged-base DNA sequencing readout on waveguide 108a-m surfaces, is illustrated. At step 305, method 300 includes immobilizing, on a surface of a waveguide 108a-m, a nucleotide fragment 104a-n. As used in this disclosure, a “nucleotide fragment 104a-n” is a sequence of two or more nucleotides, as described above, to be replicated and/or sequenced. First fragment may include a fragment of a nucleotide sample. As used in this disclosure, a “nucleotide sample” includes, without limitation, any sequence of DNA and/or RNA covered from a biological organism or portion thereof. Biological organism may include, without limitation, representatives of any kingdom of organisms, including animals, pants, fungi, archaea, bacteria, protists, and/or monera. A biological organism may also include any kind of virus or phage. A biological organism may include any object including self-replicating nucleotide sequence, where “self-replicating” indicates an ability to make copies of itself without assistance of artificial or mechanized processes.

With further reference to FIG. 3, method 300 may include one or more processes for extraction, purification, and/or amplification of nucleotide samples. Extraction may include, for instance, collection of fluid and/or tissue samples from organisms and/or environments containing a biological organism or portion thereof from which genetic material is to be extracted. Extraction may include one or more steps of lysing or otherwise opening elements containing genetic material, such as lysing cell walls and/or membranes, lysing a nuclear membrane, or the like. Extraction may include performance of one or more steps to separate genetic material from other material, including without limitation separation by density using a centrifuge; separation steps may be alternated with lysing steps.

Still referring to FIG. 1, preparation of nucleotide fragment 104a-n may include restriction and/or division of a nucleotide sample into fragments. Division may include, without limitation restriction, using one or more restriction enzymes, which may divide a nucleotide sample at one or more target points such as particular nucleotides and/or sequences thereof. Division may include division using clustered regularly interspaced short palindromic repeats (CRISPR), which may include a family of DNA sequences, for example as found in genomes of prokaryotic organisms. In some cases, CRISPR sequences may be derived from DNA fragments of bacteriophages that had previously infected a prokaryote. In some cases, CRIPT enzymes may be used to detect and destroy DNA from similar bacteriophages during subsequent infections. An exemplary CRISPR enzyme, Cas9 (or “CRISPR-associated protein 9”) is an enzyme that may use CRISPR sequences as a guide to recognize and cleave specific strands of DNA that are complementary to the CRISPR sequence. Cas9 enzymes together with CRISPR sequences can be used to edit genes within organisms. This editing process has a wide variety of applications including basic biological research, development of biotechnological products, and treatment of diseases.

Still referring to FIG. 3, obtaining and/or immobilizing first fragment may include, without limitation, dividing a nucleotide sample into a plurality of fragments and selecting the fragment from the plurality of fragments. Selection may include, without limitation, any step that separates fragments from one another by length.

As used in this disclosure, and with further reference to FIG. 3, “immobilization” refers to a process permitting no substantially relative movement between two relata, for example at least a probe and a surface. In some cases, a surface on which immobilization may occur may be functionalized. For example, a surface may be coated or otherwise treated in order to facilitate bonding, such as without limitation covalent bonding. In some exemplary embodiments, a surface may be functionalized with streptavidin and/or avidin and at least a probe may comprise biotin, thereby facilitating immobilization. For instance, Avidin and other biotin-binding proteins, including Streptavidin and NeutrAvidin protein, have an ability to bind up to biotin molecules, thereby facilitating immobilization. The Avidin-biotin complex is a strong non-covalent interaction (K_{d}=10⁻¹⁵M) between a protein and ligand. Bond formation between biotin and Avidin can be very rapid, and once formed, may be unaffected by extremes of pH, temperature, organic solvents and other denaturing agents. These features of biotin and Avidin—features that are shared by Streptavidin and NeutrAvidin Protein—are useful for immobilization.

Continuing to refer to FIG. 3, preparation and immobilization of nucleotide fragment 104a-n may include locally amplifying the nucleotide fragment 104a-n, where “local amplification” is defined as amplification in a localized cluster of identical fragments which may be immobilized adjacent to the nucleotide fragment 104a-n. Local amplification may be performed, without limitation, using a bridge PCR process whereby a cluster of forward and reverse copies of a fragment of interest are connected with bridging nucleotide strands forming arch-like structures on a surface, removal and/or severing of bridging strands using a targeted cleavage technique such as without limitation CRISPR, and removal of reverse copies. Persons skilled in the art, upon reviewing the entirety of this disclosure, will be aware of various ways in which local amplification may be accomplished. Local amplification may result in generation of a cluster of identical nucleotide fragments 104a-n immobilized on the surface of the waveguide 108a-m as a function of the local amplification. Each step described in this disclosure as performed on nucleotide fragment 104a-n may be performed in parallel on each fragment in a cluster of identical fragments; in an embodiment, optical signature of a capping agent a-n and/or signature element 120a-n may be amplified by simultaneous presence of such capping agent a-n and/or signature element 120a-n on a plurality of fragments in a cluster after a given addition step.

Further referring to FIG. 3, one or more adaptors may be appended to one or more ends of nucleotide fragment. Additional motifs may be introduced using reduced cycle amplification, including without limitation any binding locus as described above, a sequence to match to an initializing oligonucleotide, oligonucleobase, or the like.

Still referring to FIG. 3, immobilization may include immobilization on a surface of a waveguide 108a-m. As used in this disclosure, a “waveguide” is an element configured for propagation of electromagnetic (“EM”) waves. In some cases, a waveguide 108a-m may be configured to propagate an EM wave by any of total internal reflection, attenuated total internal reflection, and/or frustrated internal reflection. In some cases, a waveguide 108a-m may be configured to propagate an EM wave through reflection, transmission, and/or scattering. In some cases, waveguide 108a-m may be configured to propagate EMR through surface plasmons, for example without limitation through surface plasmon resonance (SPR). SPR may include resonant oscillation of conduction electrons, for instance at an interface between negative and positive permittivity material stimulated by incident light. SPR may alternatively or additionally be used to measure adsorption of material onto planar metal, such as without limitation gold or silver, surfaces or onto a surface of metal nanoparticles. In some cases, waveguide 108a-m may include an optical waveguide 108a-m configured to propagate EM waves, such as without limitation light by way of total internal reflection. Accordingly, in some cases, waveguide 108a-m may have an index of refraction that is substantially greater than a medium surrounding waveguide 108a-m. For example, in some exemplary cases, waveguide 108a-m may comprise sapphire and have an index of refraction which is greater than 2 and medium surrounding the waveguide 108a-m may comprise water and have an index of refraction of about 1.33.

Further referring to FIG. 3, waveguide 108a-m may include any structure that may guide waves, such as electromagnetic waves or sound waves, by restricting at least a direction of propagation of the waves. Waves in open space may propagate in multiple directions, for instance in a spherical distribution from a point source. A waveguide 108a-m may confine a wave to propagate in a restricted sent of directions, such as propagation in one dimension, one direction, or the like, so that the wave does not lose power, for instance to the inverse-square law, while propagating, and/or so that the wave is directed to a desired destination such as a sensor, light detector 132a-m, or the like. In an embodiment, a waveguide 108a-m may exploit total reflection at walls, confining waves to the interior of a waveguide 108a-m. For example, waveguide 108a-m may include a hollow conductive metal pipe used to carry high frequency radio waves such as microwaves. Waveguide 108a-m may include optical waveguides 108a-m that when used at optical frequencies are dielectric waveguides 108a-m whereby a structure with a dielectric material with high permittivity and thus a high index of refraction may be surrounded by a material with a material with lower permittivity. Such a waveguide 108a-m may include an optical fiber, such as used in fiberoptic devices or conduits. Optical fiber may include a flexible transparent fiber made from silica or plastic that includes a core surrounded by a transparent cladding material with a lower index of refraction. Light may be kept in the core of the optical fiber by the phenomenon of total internal reflection which may cause the fiber to act as a waveguide 108a-m. Fibers may include both single-mode and multi-mode fibers. Acting as a waveguide 108a-m, fibers may support one or more finite transverse modes by which light can propagate along the fiber. Waveguides 108a-m may be made from materials such as silica, fluorozirconate, fluoroaluminate, chalcogenide glass, sapphire, fluoride, silicon, silicon nitride and/or plastic. Sensors placed along waveguide 108a-m may include any photon detector 132a-m. In some cases, an output of optical waveguide 108a-m may be coupled to a photodetector 132a-m as described in further detail below.

In an embodiment, and with further reference to FIG. 3, a waveguide 108a-m making up and/or connected with surface may be composed at least in part of silicon nitride (Si₃N₄), silicon dioxide (SiO₂), and/or any other material suitable for transmission and/or internal reflection of visible, ultraviolet, and/or near-infrared wavelengths. Waveguide 108a-m may be composed at least in part of material having a broad transmission window, defined as a window permitting transmission of all fluorescent and/or absorbance wavelengths, as described in this disclosure, to be used in detection of optical signatures, with low levels of attenuation.

Still referring to FIG. 3, at step 310, method 300 includes exposing nucleotide fragment 104a-n to a first plurality of capped nucleotides a-n. Capped nucleotides a-n may include any capped nucleotides a-n as defined above. In an embodiment, first plurality of capped nucleotides a-n includes a first plurality of nucleotide types, such as a solution of nucleotides, dNTPs, and/or NTPs containing roughly equal quantities of adenine, cytosine, guanine, thymine, and/or uracil, or dNTPs and/or NTPs thereof. In an embodiment, each distinct nucleotide type has a distinct capping agent a-n; for instance, all adenine nucleotides, NTPs, and/or dNTPs may have a first type of capping agent a-n, all cytosine NTPs, and/or dNTPs may have a second type of capping agent a-n, all guanine NTPs, and/or dNTPs may have a third type of capping agent a-n, and all thymine and/or uracil NTPs, and/or dNTPs may have a fourth type of capping agent a-n. In other words, first plurality of nucleotide types may include all deoxyribonucleic and/or ribonucleic acid nucleotide types.

Continuing to refer to FIG. 3, each distinct capping agent a-n may have a distinct optical signature; this may be implemented without limitation as described above. For instance, each type of capping agent a-n may have a signature element 120a-n that produces an optical signature that affects a wavelength, range of wavelengths, and/or set of wavelengths which is detectably different from a wavelength, range of wavelengths, and/or set of wavelengths affected by each optical signature produced by each signature element 120a-n of each other type of capping agent a-n. Each or any distinct optical signature may include a fluorescent signature. As used in this disclosure, a “fluorescent signature” is a signature characterized by fluorescence in an identifiable wavelength, range of wavelengths, and/or set of wavelengths. Each or any distinct optical signature may include an absorption signature. As used in this disclosure, an “absorbance signature” is a signature characterized by absorption of an identifiable wavelength, range of wavelengths, and/or set of wavelengths.

Still referring to FIG. 3, nucleotide fragment 104a-n may include a binding locus 124, which may include any binding locus 124 as described above in reference to FIGS. 1-2. Binding locus 124 may be adjacent to and/or attached to the fragment. Binding locus 124 may be at a proximal end of nucleotide fragment 104a-n, where the proximal end is the end attached to the substrate and/or waveguide 108a-m. Binding locus 124 may be attached to and/or at substrate and/or waveguide 108a-m. Binding locus 124 may alternatively be at a distal end of nucleotide fragment 104a-n, where the distal end is the end opposite proximal end.

Further referring to FIG. 3, an enzyme that facilitates covalent bonds between nucleotides, such as a polymerase as described above, may be introduced. Enzyme may attach a single capped nucleotide to binding locus 124; single capped nucleotide may be located at binding locus 124 by virtue of a base pairing with a nucleotide of nucleotide fragment 104a-n that is at and/or adjacent to binding locus 124. As a result, single capped nucleotide may complement a first nucleotide of nucleotide sequence, such that detection of an optical signature of the single capped nucleotide may be used to determine an identity of the first nucleotide. Covalent bonds between other capped nucleotides a-n may be blocked by capping agents a-n, ensuring that only a single capped nucleotide is added.

Still referring to FIG. 3, at step 315, method 300 may include severing base pair connections between the at least a nucleotide fragment 104a-n and the first plurality of capped nucleotides a-n. In an embodiment, when plurality of capped nucleotides a-n are added to and/or exposed to nucleotide fragment 104a-n, multiple capped nucleotides a-n may form base pairs with nucleotide fragment 104a-n. Severing base pare connections may free such capped nucleotides a-n from nucleotide fragment 104a-n, such that they may be washed away. Severing may be performed using any process suitable for severing base pair connections in PCR processes or the like, including without limitation introduction of an enzyme that severs base pairs, heating to a threshold temperature that denatures base pairs, or the like; mechanism for severing may be kept active until all or substantially all loose capped nucleotides a-n are washed and/or rinsed away. In an embodiment, nucleotide fragment 104a-n remains attached, meaning that nucleotide fragment 104a-n remains intact and continues to be immobilized on the waveguide 108a-m; all fragments in a cluster of fragments created by local amplification may similarly remain attached, after severing of base pairs.

With further reference to FIG. 3, upon severing base pair connections, a first single nucleotide, of the first plurality of capped nucleotides a-n, may remain immobilized at nucleotide binding locus 124 adjacent to first nucleotide sequence. In other words, first single nucleotide may have formed a base pair with the nucleotide in the immobilized fragment that is closest to binding locus 124, and may have formed a covalent bond or otherwise been immobilized to binding locus 124, such that first capped nucleotide remains at such location after severing and removal of other capped nucleotides a-n; this may be true of all or substantially all fragments in a cluster of fragments created using local amplification.

Still referring to FIG. 3, at step 320, method 300 includes detecting a first distinct optical signature of a first distinct capping agent a-n of the first single nucleotide using the waveguide 108a-m; this may be performed, without limitation, in any manner described in this disclosure. In an embodiment, where first distinct optical signature is a fluorescent signature, detection may include excitation of fluorescent material of and/or attached to capping agent a-n using a light source, causing the fluorescent material to fluoresce, giving off a signature wavelength of light. For instance, detection may include exciting first distinct capping agent a-n using an out of plane light source 128 and detecting the fluorescent signature as a function of the exciting, for instance and without limitation as described above. Alternatively or additionally, detecting first distinct optical signature may include exciting first distinct capping agent a-n using a waveguide light source and detecting the fluorescent signature as a function of the exciting. A “waveguide light source,” as used in this disclosure is a light source that provides light to fragments and/or capping agent a-n by means of waveguide 108a-m.

Still referring to FIG. 3, detecting first distinct optical signature may include propagating, using the waveguide 108a-m, an evanescent wave from the surface, and detecting the first distinct optical signature as a function of the evanescent wave. As used in this disclosure, an “evanescent wave,” which may also be referred to as an “evanescent field,” is a wave that exhibits a rapidly decaying (or vanishing) field amplitude in a certain spatial direction, for example orthogonal to surface of waveguide 108a-m. In some cases, an evanescent wave may not contribute to energy transport in a spatial direction such as a direction in which evanescent wave exhibits a rapidly decaying or vanishing field amplitude, although in some cases a Poynting vector (averaged over one oscillation cycle) may have non-zero components in other directions. Evanescent wave may be used to excite and/or be absorbed by signature element 120a-n. In some cases, a light signal detected by a sensor as described below may indicate presence and/or absence of a given signature element 120a-n. For example, a light signal attenuated for a given wavelength, range of wavelengths, and/or set of wavelengths may indicate that a signature element 120a-n absorbing the wavelength, range of wavelengths, and/or set of wavelengths is present, as the attenuated light signal may result from evanescent wave coupling, for instance via absorption, into signature element 120a-n. As an evanescent wave “vanishes” along a certain direction, its field amplitude, and therefore ability to be used for sensing, may diminish drastically as distance away from surface increases. For example, depending upon parameters, such as index of refraction, light wavelength, light coupling angle, to name a few, an evanescent wave may practically propagate less than 100 μm from surface, less than 10 μm from surface, or even less than 1 μm from service.

For example, and with further reference to FIG. 3, an optical system may include multiple ring resonators and/or microring resonators where each ring resonator may be functionalized with a different signature element and/or may be functionalized with a polymerase and/or modified polymerase. In an embodiment, a “microring resonators” is a type of whispering gallery mode sensor capable of detecting bulk changes in refractive index. A microring resonator may include a set of waveguides in which at least one is a closed loop coupled to some sort of light input and output. When light of a resonant wavelength is passed through the loop from input waveguide, may build up in intensity over multiple round-trips due to constructive interference and may be output to an output bus waveguide which may serve as a detector waveguide. Signature element may thus be detected using a change to refractive index as detected using a microring resonator. Alternatively or additionally, any method or combination of surface methods (e.g. electrical and/or optical) may be used to detect optical signature, including without limitation transistors, nanopores, surface plasmon resonant thin films or particles, surfaces used for SERS spectroscopy, and/or electrical resistance-based sensors.

With continued reference to FIG. 3, where first distinct optical signature is an absorption signature, detecting first distinct optical signature may include passing a broadband light through the waveguide 108a-m and determining an attenuation frequency of the broadband light, where an “attenuation frequency” is a frequency absorbed and/or attenuated by a signature element 120a-n as described above. Passing broadband light through the waveguide 108a-m may include generating the broadband light using a tunable laser.

Still referring to FIG. 3, general sensing techniques that may be employed in detection of optical signature may include but are not limited to using a doped optical waveguide 108a-m or electrodes near a waveguide 108a-m to sense an optical change or resistance change, respectively, after at least a probe has been cleaved. In some examples, optical changes may be detected using surface plasmon resonances, Mach-Zehnder interferometers, spiral waveguides 108a-m, Bragg gratings, and/or photonic crystals or magnetic dielectric mirrors. Detecting a first distinct optical signature may include detecting the first distinct optical signature using at least a spectrometer, for instance and without limitation as described below.

With continued reference to FIG. 3, method 300 may include removing first distinct capping agent a-n from first single nucleotide. Removing first distinct capping agent a-n may include irradiating the first distinct capping agent a-n; this may be performed using any light source, and/or any mechanism of delivery, including without limitation using waveguide 108a-m. As another non-limiting example, removing first distinct capping agent a-n may include removing the first distinct capping agent a-n using a chemical agent. Capping agent a-n may be removed chemically and/or by light, for instance and without limitation using a light-catalyzed reaction. In an embodiment, light used in a light-catalyzed de-capping process for a capping agent a-n of a given nucleotide may have a distinct wavelength from light used to catalyze attachment of the given nucleotide; this may allow controlled and sequential attachment and de-capping to be performed, which may, as a non-limiting example, permit performance of detection steps, as described below, between attachment and de-capping steps. Chemical catalysis may be performed using any suitable chemical catalyst that may occur to persons skilled in the art upon reviewing the entirety of this disclosure, including without limitation enzymes, CRISPR enzymes, or the like; a chemical agent used to catalyze cap removal for a cap of a given nucleotide may be distinct from a chemical catalyst used to attach that nucleotide, which may, as a non-limiting example, ensure that de-capping and deposition may be controlled separately, permitting intervening sensing or other steps. Any other modality may be used to trigger and/or catalyze removal, including without limitation local and/or overall heating at the reaction and/or deposition site. More generally, controlled decapping of capping agents a-n may be activated in response to various triggers ranging from biological conditions such as without limitation pH, reactive oxygen, or the like to external stimuli such as without limitation magnetic, thermal, electrical, and/or other stimuli.

Still referring to FIG. 3, nucleotide addition, detection, and cap-removal steps as described above may be performed iteratively. For instance, method 100 may include performing at least a detection iteration. In an embodiment, each detection iteration may include any steps described above. For instance, each detection iteration may include exposing nucleotide fragment 104a-n to a second plurality of capped nucleotides a-n, which may include any or all capped nucleotides a-n suitable for use in first plurality of capped nucleotides a-n as described above. A detection iteration may include severing base pair connections between the at least a nucleotide fragment 104a-n and the second plurality of capped nucleotides a-n. In an embodiment, nucleotide fragment 104a-n may remain attached as before. A second single nucleotide of the second plurality of capped nucleotides a-n, may remain chained to the first single nucleotide, where “chained to the first single nucleotide” means incorporated in a sequence of two or more nucleotides linked by RNA, DNA, and/or a peptide or peptide nucleic acid backbone that includes first single nucleotide. A detection iteration may include detecting a second distinct optical signature of a second distinct capping agent a-n of the second single nucleotide using the waveguide 108a-m. In an embodiment, each detection iteration may be performed after removal of a capping agent a-n from a previous detection iteration.

Further referring to FIG. 3, each and/or any step described above may be followed in some embodiments by washing, which may include any washing step or modality described below. Washing may be performed, without limitation, prior to detection procedures and/or steps as described below; in some embodiments, this may prevent or make less probable false positive detections of labels and/or agents indicative of nucleotide attachment by removing any free-floating nucleotides that have not attached.

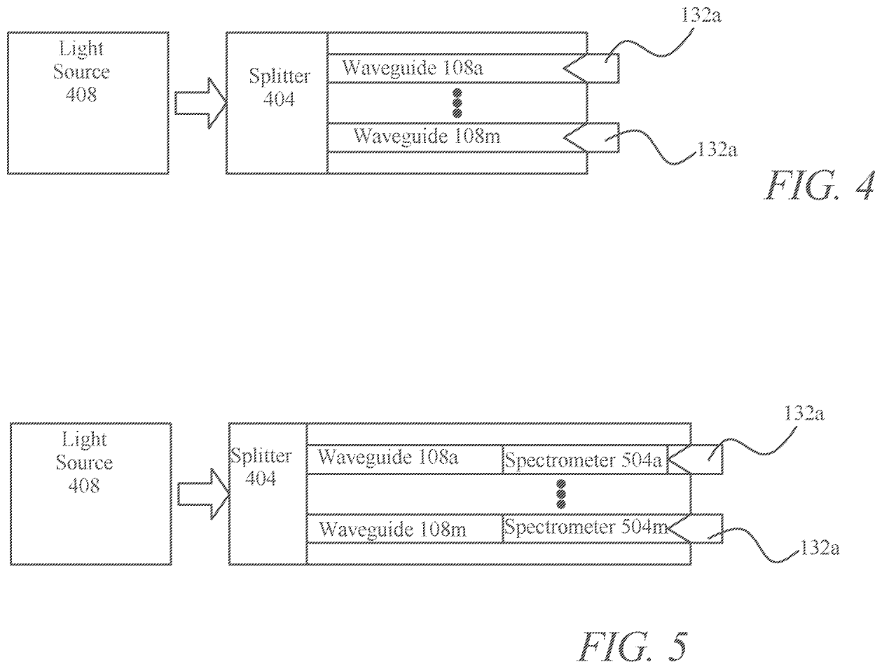

Referring now to FIG. 4, an exemplary embodiment of optics for detection of optical signature, such as without limitation a fluorescence and/or absorbance-based is illustrated. In this example, a splitter 404 may be used to direct light from a single light source 408 into a plurality of separate waveguides 108a-m used as described above. Each waveguide 108a-m may have an independent photodetector 132a-m; photodetector 132a-m may be used to detect the loss of light through the waveguide 108a-m due to absorption by the optical absorber. For instance, and without limitation, a light source may be swept through a range of wavelengths, and times at which attenuation is detected by photodetectors 132a-m may be linked by analytical circuitry as described below to a wavelength being emitted at the moment of attenuation, permitting identification of attenuated wavelength. Alternatively or additionally, photodetectors 132a-m may be preceded by spectrometers and/or filters as described in further detail below.

Still referring to FIG. 4, photodetectors 132a-m may include, without limitation, Avalanche Photodiodes (APDs), Single Photon Avalanche Diodes (SPADs), Silicon Photomultipliers (SiPMs), Photo-Multiplier Tubes (PMTs), Micro-Channel Plates (MCPs), Micro-Channel Plate Photomultiplier Tubes (MCP-PMTs), Indium gallium arsenide semiconductors (InGaAs), photodiodes, and/or photosensitive or photon-detecting circuit elements, semiconductors and/or transducers. Avalanche Photo Diodes (APDs), as used herein, are diodes (e.g. without limitation p-n, p-i-n, and others) reverse biased such that a single photon generated carrier can trigger a short, temporary “avalanche” of photocurrent on the order of milliamps or more caused by electrons being accelerated through a high field region of the diode and impact ionizing covalent bonds in the bulk material, these in turn triggering greater impact ionization of electron-hole pairs. APDs provide a built-in stage of gain through avalanche multiplication. When the reverse bias is less than the breakdown voltage, the gain of the APD is approximately linear. For silicon APDs this gain is on the order of 10-100. Material of APD may contribute to gains. Germanium APDs may detect infrared out to a wavelength of 1.7 micrometers. InGaAs may detect infrared out to a wavelength of 1.6 micrometers. Mercury Cadmium Telluride (HgCdTe) may detect infrared out to a wavelength of 14 micrometers. An APD reverse biased significantly above the breakdown voltage is referred to as a Single Photon Avalanche Diode, or SPAD. In this case the n-p electric field is sufficiently high to sustain an avalanche of current with a single photon, hence referred to as “Geiger mode.” This avalanche current rises rapidly (sub-nanosecond), such that detection of the avalanche current can be used to approximate the arrival time of the incident photon. The SPAD may be pulled below breakdown voltage once triggered in order to reset or quench the avalanche current before another photon may be detected, as while the avalanche current is active carriers from additional photons may have a negligible effect on the current in the diode. At least a first photodetector 132a-m may be configured to generate a first electrical multiplication output signal as a function of the first matrix multiplication output, where first electrical multiplication signal may include without limitation any voltage and/or current waveform generated in response to detection of first matrix multiplication output.

Referring now to FIG. 5, another example of a system diagram of optics in an absorbance-based detection scheme is illustrated. In this example, a broadband LED or other broadband source may be used to transmit light to one or more waveguides 108a-m; a plurality of waveguides 108a-m may be provided light via a splitter as described above. One or more photodetectors 132a-m and/or arrays thereof may be preceded in an optical path from signature element 120a-n and waveguide 108a-m by a spectrometer 504a-m. Spectrometer 504a-m may be on-chip or off-chip. For instance, and without limitation, spectrometer 504a-m may include a reflective and/or refractive grating that separates light by wavelength such that a location of a photodetector 132a-m in a photodetector 132a-m array on which light is cast by the grating determines a frequency and/or wavelength of light cast thereon; in this way, levels of light determined by each such photodetector 132a-m may indicate which frequency and/or frequencies are attenuated.

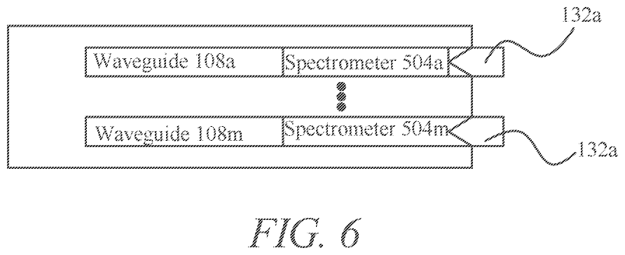

Referring now to FIG. 6, an exemplary embodiment of optics in a fluorescence-based detection scheme using a spectrometer 504a-m (on-chip or off-chip) before the photodetector 132a-m is illustrated. Light may be generated as before by an out-of-plane light and/or by a light that transmits through waveguide 108a-m. Spectrometer 504a-m may detect a transmitted wavelength and/or spread wavelengths on a detector 132a-m array as described above, enabling detection according to position.

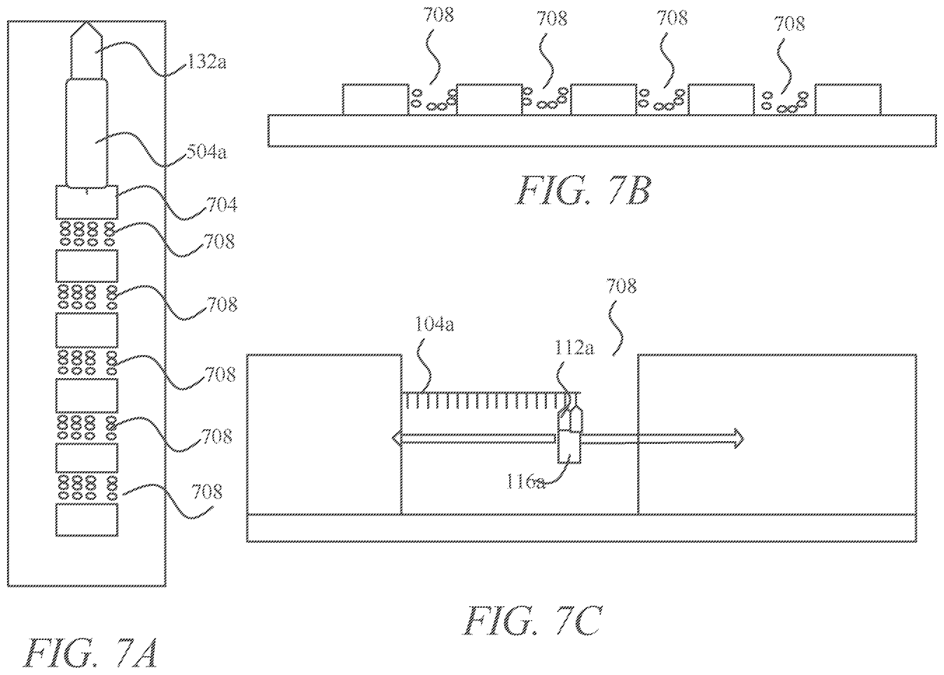

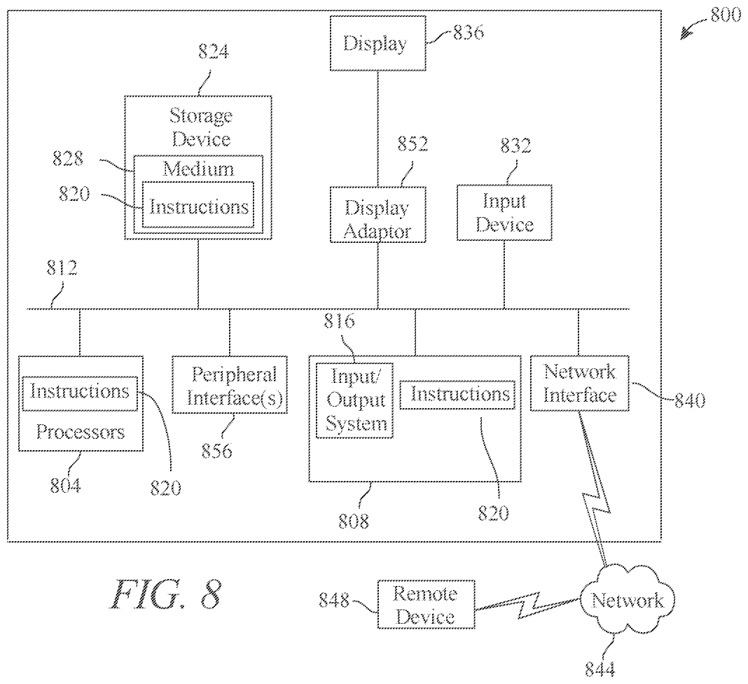

Referring now to FIG. 7A, in some implementations waveguide 108a-m may be a subwavelength grating-type waveguide 704 in order to increase optical interaction of a reaction with the waveguide 704. Grating-type waveguide 704 may have a plurality of wells 708 in which nucleotide fragments may be immobilized, and in which capped nucleotides may be added. If fluorescence is used to label and track the added nucleotides, then the emission may be more likely to be captured in the waveguide 704 if the emitter is within the guided mode region and/or well 708. FIG. 7B shows a side view of grating 704 with wells. As shown in the exemplary detail of FIG. 7C, each well 708 may include one or more immobilized fragments 104a to which capped nucleotides 112a may be added. Optical signature light may be captured at well walls and transferred via waveguide to detection optics and/or spectrometers. Absorbance may also be higher if the absorber is within the central region of the guided mode in the waveguide 108a-m. In some implementations, multiple waveguides 108a-m may converge onto a single spectrometer 504a-m or photodetector 132a-m or both in order to capture more fluorescence or to increase overall absorbance. In an embodiment, grating used in this manner may capture a greater proportion of emitted light, and/or otherwise aid in isolating localized effects on light that is transmitted, blocked, reflected, fluoresced or scattered as part of optical signature.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random-access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

FIG. 8 shows a diagrammatic representation of one embodiment of a computing device in the exemplary form of a computer system 800 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 800 includes a processor 804 and a memory 808 that communicate with each other, and with other components, via a bus 812. Bus 812 may include any of several types of bus structures including, but not limited to, a memory bus, a memory controller, a peripheral bus, a local bus, and any combinations thereof, using any of a variety of bus architectures.

Processor 804 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 804 may be organized according to Von Neumann and/or Harvard architecture as a non-limiting example. Processor 804 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating-point unit (FPU), and/or system on a chip (SoC).

Memory 808 may include various components (e.g., machine-readable media) including, but not limited to, a random-access memory component, a read only component, and any combinations thereof. In one example, a basic input/output system 816 (BIOS), including basic routines that help to transfer information between elements within computer system 800, such as during start-up, may be stored in memory 808. Memory 808 may also include (e.g., stored on one or more machine-readable media) instructions (e.g., software) 820 embodying any one or more of the aspects and/or methodologies of the present disclosure. In another example, memory 808 may further include any number of program modules including, but not limited to, an operating system, one or more application programs, other program modules, program data, and any combinations thereof.

Computer system 800 may also include a storage device 824. Examples of a storage device (e.g., storage device 824) include, but are not limited to, a hard disk drive, a magnetic disk drive, an optical disc drive in combination with an optical medium, a solid-state memory device, and any combinations thereof. Storage device 824 may be connected to bus 812 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 824 (or one or more components thereof) may be removably interfaced with computer system 800 (e.g., via an external port connector (not shown)). Particularly, storage device 824 and an associated machine-readable medium 828 may provide nonvolatile and/or volatile storage of machine-readable instructions, data structures, program modules, and/or other data for computer system 800. In one example, software 820 may reside, completely or partially, within machine-readable medium 828. In another example, software 820 may reside, completely or partially, within processor 804.

Computer system 800 may also include an input device 832. In one example, a user of computer system 800 may enter commands and/or other information into computer system 800 via input device 832. Examples of an input device 832 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 832 may be interfaced to bus 812 via any of a variety of interfaces (not shown) including, but not limited to, a serial interface, a parallel interface, a game port, a USB interface, a FIREWIRE interface, a direct interface to bus 812, and any combinations thereof. Input device 832 may include a touch screen interface that may be a part of or separate from display 836, discussed further below. Input device 832 may be utilized as a user selection device for selecting one or more graphical representations in a graphical interface as described above.

A user may also input commands and/or other information to computer system 800 via storage device 824 (e.g., a removable disk drive, a flash drive, etc.) and/or network interface device 840. A network interface device, such as network interface device 840, may be utilized for connecting computer system 800 to one or more of a variety of networks, such as network 844, and one or more remote devices 848 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 844, may employ a wired and/or a wireless mode of communication. In general, any network topology may be used. Information (e.g., data, software 820, etc.) may be communicated to and/or from computer system 800 via network interface device 840.

Computer system 800 may further include a video display adapter 852 for communicating a displayable image to a display device, such as display device 836. Examples of a display device include, but are not limited to, a liquid crystal display (LCD), a cathode ray tube (CRT), a plasma display, a light emitting diode (LED) display, and any combinations thereof. Display adapter 852 and display device 836 may be utilized in combination with processor 804 to provide graphical representations of aspects of the present disclosure. In addition to a display device, computer system 800 may include one or more other peripheral output devices including, but not limited to, an audio speaker, a printer, and any combinations thereof. Such peripheral output devices may be connected to bus 812 via a peripheral interface 856. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A method of tagged-base DNA sequencing readout on waveguide surfaces, the method comprising:
immobilizing, a surface of a waveguide, a nucleotide fragment;
exposing the nucleotide fragment to a first plurality of capped nucleotides, wherein:
the first plurality of capped nucleotides include a first plurality of nucleotide types;
each distinct nucleotide type has a distinct capping agent; and
each distinct capping agent has a distinct optical signature;
severing base pair connections between the at least a nucleotide fragment and the first plurality of capped nucleotides, wherein:
the nucleotide fragment remains attached; and
a first single nucleotide, of the first plurality of capped nucleotides, remains immobilized on a nucleotide binding locus adjacent to the first nucleotide sequence; and
detecting a first distinct optical signature of a first distinct capping agent of the first single nucleotide using the waveguide.

2. The method of claim 1, wherein the first fragment is a fragment of a nucleotide sample.

3. The method of claim 2, wherein immobilizing the first fragment further comprises:
dividing the nucleotide sample into a plurality of fragments; and
selecting the first fragment from the plurality of fragments.

4. The method of claim 1, wherein the first distinct optical signature is a fluorescent signature.

5. The method of claim 1, wherein detecting the first distinct optical signature further comprises:
exciting the first distinct capping agent using an out of plane light source; and
detecting the fluorescent signature as a function of the exciting.

6. The method of claim 1, wherein detecting the first distinct optical signature further comprises:
exciting the first distinct capping agent using a waveguide light source; and
detecting the fluorescent signature as a function of the exciting.

7. The method of claim 1, wherein the first distinct optical signature is an absorption signature.

8. The method of claim 5, wherein detecting the first distinct optical signature further comprises:
passing a broadband light through the waveguide; and
determining an attenuation frequency of the broadband light.

9. The method of claim 6, wherein the waveguide is composed of material having a broad transmission window.

10. The method of claim 6, wherein passing the broadband light through the waveguide further comprises generating the broadband light using a tunable laser.

11. The method of claim 1, wherein detecting a first distinct optical signature further comprises detecting the first distinct optical signature using at least a spectrometer.

12. The method of claim 1, wherein detecting the first distinct optical signature further comprises propagating, using the waveguide, an evanescent wave from the surface, and detecting the first distinct optical signature as a function of the evanescent wave.

13. The method of claim 1 further comprising removing the first distinct capping agent from the first single nucleotide.

14. The method of claim 14, wherein removing the first distinct capping agent further comprises irradiating the first distinct capping agent using the waveguide.

15. The method of claim 14, wherein removing the first distinct capping agent further comprises removing the first distinct capping agent using a chemical agent.

16. The method of claim 14 further comprising performing at least a detection iteration, wherein each detection iteration further comprises:
exposing the nucleotide fragment to a second plurality of capped nucleotides;
severing base pair connections between the at least a nucleotide fragment and the second plurality of capped nucleotides, wherein:
the nucleotide fragment remains attached; and
a second single nucleotide, of the second plurality of capped nucleotides, remains chained to the first single nucleotide; and
detecting a second distinct optical signature of a second distinct capping agent of the second single nucleotide using the waveguide.

17. The method of claim 17, wherein each detection iteration is performed after removal of a capping agent from a previous detection iteration.

18. The method of claim 1 further comprising:
locally amplifying the nucleotide fragment; and
generating a cluster of identical nucleotide fragments immobilized on the surface of the waveguide as a function of the local amplification.

19. The method of claim 1, wherein the first plurality of nucleotide types includes all deoxyribonucleic acid nucleotide types.

20. The method of claim 1, wherein the first plurality of nucleotide types includes all ribonucleic acid nucleotide types.
